(19) United States
(12) Patent Application Publication
(10) Pub. No.: US 2013/0330349 A1
(43) Pub. Date: Dec. 12, 2013

(54) HIGH AFFINITY MOLECULES CAPABLE OF BINDING A TYPE A PLEXIN RECEPTOR AND USES OF SAME

(71) Applicants: Gera Neufeld, Haifa (IL); Boaz Kigel, Kiryat-Tivon (IL); Noa Rabinowicz, Haifa (IL); Asya Varshavsky, Haifa (IL); Ofra Kessler, Haifa (IL)

(72) Inventors: Gera Neufeld, Haifa (IL); Boaz Kigel, Kiryat-Tivon (IL); Noa Rabinowicz, Haifa (IL); Asya Varshavsky, Haifa (IL); Ofra Kessler, Haifa (IL)

(73) Assignee: Rappaport Family Institute for Research in the Medical Sciences, Haifa (IL)

(21) Appl. No.: 14/000,914

(22) PCT Filed: Feb. 23, 2012
(86) PCT No.: PCT/IL12/50057

§ 371 Date: Aug. 22, 2013

Related U.S. Application Data
(60) Provisional application No. 61/445,567, filed on Feb. 23, 2011.

(51) Int. Cl. C07K 16/28; A61K 39/395

(52) U.S. Cl. CPC C07K 16/2863; A61K 39/39558
USPC 424/139.1; 530/387.3; 530/387.9; 435/375

(57) ABSTRACT

A high affinity molecule is provided. The high affinity molecule comprises a binding domain which binds a type-A plexin receptor, wherein said binding domain inhibits proliferative signals through said type-A plexin receptor but does not interfere with binding of a neuropilin or semaphorin 6A to said type-A plexin receptor.

### FIELD AND BACKGROUND OF THE INVENTION

The present invention, in some embodiments thereof, relates to high affinity molecules capable of binding a type A plexin receptor and uses of same.

The human plexin gene family comprises at least nine members in four subfamilies.

The extracellular domains of plexins encompasses about 500 amino acid semaphorin domains. The highly conserved cytoplasmic moieties of plexins (about 600 amino acids), however share no homology with any other known protein.

Plexin-B1 is a receptor for the transmembrane semaphorin Sema4D (CD 100), and plexin-C1 is a receptor for the GPI-anchored semaphorin Sema7A (Sema-K1). Type A plexins tranduce class-6 semaphorin signaling and also interact with neuropilins as co-receptors and tranduce the signal of class 3 semaphorins.

The human gene related to the class 6 semaphorin family termed semaphorin 6B or SEMA6B was cloned in 2001 by Correa wt al. (Genomics, 2001, 1:73(3):343-8. Two splice variants of this gene were identified. This protein signals by interacting with Plexin A4. The gene was found to be expressed in neural tissues.

WO 2001/14420 teaches compositions and methods related to newly isolated plexins. Plexin specific binding agents are disclosed and their use in the treatment of oncological diseases is envisaged. Specifically disclosed is the nucleic acid sequence and amino acid sequence of plexin A4. WO 2001/14420 also contemplates suppressing or altering aberrant cell growth involving a signaling between plexin and neuropilin using an agent (e.g., an antibody) which interferes with the binding between a plexin and a neuropilin.

U.S. Patent Application 20060228710 provides a comprehensive list of molecular targets, such as semaphorin 6B, which can be used in the diagnosis and treatment of cancer.

U.S. Patent Application 20060127902 discloses a method of treating glioma using an anti semaphorin 6B antibody.

WO 2007000672 discloses peptidic antagonists of class III sempahorins/neuropilins complexes comprising an amino acid sequence which is derived from the transmembrane domain of plexin-A4 and uses thereof in the treatment of diseases associated with abnormal angiogenesis.

### SUMMARY OF THE INVENTION

According to an aspect of some embodiments of the present invention there is provided a high affinity molecule comprising a binding domain which binds a type-A plexin receptor, wherein the binding domain inhibits proliferative signals through said type-A plexin receptor but does not interfere with binding of a neuropilin or semaphorin 6A to the type-A plexin receptor.

According to an aspect of some embodiments of the present invention there is provided a composition of matter comprising at least two distinct high affinity molecules the at least two distinct high affinity molecules capable of binding and inhibiting signaling from a plexin signaling molecule selected from the group consisting of a type A plexin receptor, a semaphorin a co-receptor of the type A plexin receptor and a ligand of the co-receptor.

According to some embodiments of the invention, wherein the co-receptor is an FGFR or a VEGFR-2.

According to some embodiments of the invention, the high affinity molecule is selected from the group consisting of an antibody, a peptide, an aptamer and a small molecule.

According to some embodiments of the invention, the type-A plexin receptor comprises Plexin-A4.

According to some embodiments of the invention, the binding of the binding domain to the type-A plexin receptor comprises an affinity of at least 10⁻⁶ M.

According to some embodiments of the invention, the antibody comprises a monoclonal antibody.

According to some embodiments of the invention, the antibody comprises a bispecific antibody.

According to some embodiments of the invention, the bispecific antibody binds the type-A plexin receptor and at least one of an FGFR and semaphorin 6B.

According to some embodiments of the invention, the bispecific antibody binds a type-A1 plexin receptor and at least one of VEGFR-2 and semaphorin 6D.

According to some embodiments of the invention, the bispecific antibody binds to distinct epitopes on the type-A plexin receptor.

According to some embodiments of the invention, the high affinity binding molecule binds an epitope on an extracellular domain of the Type A plexin receptor, the extracellular domain being selected from the group consisting of a sema domain (pfam number PF01403) and an IgG domain.

According to some embodiments of the invention, the high affinity molecule induces internalization of the plexin receptor.

According to an aspect of some embodiments of the present invention there is provided an isolated antibody comprising an antigen recognition domain which binds a type A plexin receptor, wherein the antibody induces internalization of the type A plexin receptor upon binding thereto.

According to some embodiments of the invention, the type-A plexin receptor is selected from the group consisting of Plxn-A1, Plxn-A2, Plxn-A3 and Plxn-A4.

According to some embodiments of the invention, the isolated antibody binds an epitope on an extracellular domain of the Type A plexin receptor, the domain being selected from the group consisting of a sema domain (pfam number PF01403) and an IgG domain.

According to an aspect of some embodiments of the present invention there is provided a method of reducing angiogenesis in a tissue, the method comprising contacting the tissue with the high affinity molecule or composition or the antibody, thereby reducing angiogenesis in the tissue.

According to some embodiments of the invention, the contacting is effected ex-vivo.

According to some embodiments of the invention, the tissue comprises a cancer tissue.

According to an aspect of some embodiments of the present invention there is provided a method of treating an angiogenesis-related disorder in a subject in need thereof, the method comprising administering to the subject a therapeutically effective amount of the high affinity biding molecule or composition or the isolated antibody, thereby treating the angiogenesis-related disorder.

According to an aspect of some embodiments of the present invention there is provided a method of treating cancer in a subject in need thereof, the method comprising administering to the subject a therapeutically effective amount of the high affinity binding molecule or composition or the isolated antibody of claim, thereby treating cancer.

According to an aspect of some embodiments of the present invention there is provided a pharmaceutical composition comprising a pharmaceutically acceptable carrier and as an active ingredient the high affinity molecule, isolated antibody or composition.

According to some embodiments of the invention, the pharmaceutical composition further comprises a chemotherapeutic agent.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1A is a graph showing the expression levels of mRNA encoding type-A plexins as determined in HUVEC infected with non-targeted shRNA (Control) and in HUVEC in which the expression of plexin-A1 (plexA1), plexin-A3 (plexA3) or plexin-A4 (plexA4) were silenced with specific shRNAs using real-time quantitative PCR.

FIG. 1B shows micrographs of Neuropilin levels determined in cell lysates using western blot analysis.

FIGS. 2A-C show the effect of plexin silencing on cell proliferation. FIG. 2A—HUVEC expressing a non-targeted shRNA (sh-control) or HUVEC in which the expression of indicated plexins was silenced using specific indicated shRNAs were seeded in 24 well dishes (2×10⁴ cells/well). bFGF (5 ng/ml) was added or not following cell attachment. After three days adherent cells were detached and counted in a coulter counter. FIG. 2B—Incorporation of Brdu into the DNA of HUVEC expressing a non-targeted shRNA (sh-control) or HUVEC in which the expression of specific plexins was silenced using the indicated shRNAs as measured 24 h after stimulation with bFGF. FIG. 2C—representative images of BrdU staied HUVECs in which different plexins were silenced in the presence of bFGF.

FIGS. 3A-B show the effect of plexin silencing on cell assembly and morphology. FIG. 3A—HUVEC infected with lentiviruses containing non-targeted shRNA (control) or HUVEC infected with lentiviruses directing expression of shRNAs targeting plexin-A4 (sh-plexA4) or plexin-A1 (sh-plexA1) were seeded (1.2×10⁴ cells/well) on top of Matrigel. Tube formation and quantification of bifurcations in the tubular network formed were assessed at various time points. Represented photos of tube formation assay with HUVEC that were knocked down with shRNA for plexin-A1, plexin-A3 or plexin-A4. FIG. 3B—Spheroids (500 cells/spheroid) containing HUVEC expressing a non-targeted shRNA (sh-control) or HUVEC expressing a plexin-A4 targeting shRNA (sh-plexA4) were seeded on collagen and stimulated to sprout with 5 ng/ml bFGF. Shown are representative pictures of sprouting spheroids taken after 24 h.

FIG. 4 is a graphic presentation of the proliferation of lung cancer cells (in HA460, HA2009, HA188 and A549) in which expression of plexin-A4 was silenced using plexin-A4 targeting shRNA (Sh-plexA4). The proliferation of these cells was compared to the proliferation of the respective control cells expressing a non-targeted shRNA (Sh-control). 100% represents the number of adherent cells/well as counted 4 h after seeding. Cells were counted after 3 days. In the case of A549 cells the proliferation of cells in which the expression of plexin-A1 was silenced (Sh-plexA1) and of cells in which the expression of both plexins was silenced were also determined.

FIGS. 5A-D show the effect of plexin silencing on tumor cell proliferation and tumor growth in vivo. FIG. 5A—U87MG cells were infected with lentiviruses expressing non-targeted shRNA (sh-control) plexin-A4 shRNA (sh-plexA4). The effects of targeting shRNAs to plexin-A4 on mRNA levels was assessed using real time PCR. FIG. 5B—The expression of plexin-A4 was silenced using plexin-A4 targeted shRNA (Sh-plexA4) in U87MG glioblastoma cancer cells. The proliferation of these cells was compared to the proliferation of the respective control cells expressing a non-targeted shRNA (Sh-control). 100% represents the number of adherent cells/well as counted 4 h after seeding. Cells were counted after 3 days. FIGS. 5C-D—The development of tumors derived from U87MG cells that were silenced with plexin-A4 shRNA was compared with that of control cells (left panel). The tumors were excised and weighed at the end of the experiment (right panel). Each group contained 5 mice. The experiment was repeated twice with similar results.

FIGS. 6A-G show the effect of sema6B silencing on cell proliferation and morphology. FIG. 6A—Stimulation of HUVEC with sema6A inhibited the bFGF induced proliferation of the HUVEC by ˜20% and in the absence of bFGF inhibited the survival of HUVEC by ˜70%. Furthermore, sema6A also inhibited the survival and the residual bFGF induced proliferative response in HUVEC in which plexin-A4 expression was silenced. FIG. 6B—Sema6B mRNA silencing in HUVEC. The effects of shRNAs silencing of sema6B on mRNA levels was assessed using real time PCR. Actin staining of HUVEC shows a morphological change that was very reminiscent of the change produced in response to the silencing of plexin-A4 expression. FIG. 6C—Silencing sema6B inhibited ˜85% of the mitogenic effect of bFGF. FIG. 6D—To a similar extent inhibited bFGF induced phosphorylation of ERK1/2. FIG. 6E—Sema3A mRNA silencing in HUVEC. The effects of sh-sema3A on sema3A mRNA levels was assessed using real time PCR. FIG. 6F—HUVEC proliferation, with or without bFGF, of cells that were silenced with sh-sema3A or sh-control lentivirus vector. FIG. 6G—bFGF induced ERK1/2 phosphorylation of HUVEC silenced with sema3A shRNA.

FIGS. 7A-B graphs showing the effect of sema3A and sema6B silencing on cell proliferation. Silencing of sema3A and sema6B in (FIG. 7A) U87MG glioblastoma cancer cell line or (FIG. 7B) A549 lung cancer cell line was assessed using real time PCR (upper panel). The proliferation of these cells was compared to the proliferation of the respective control cells expressing a non-targeted shRNA (Sh-control). 100% represents the number of adherent cells/well as counted 4 h after seeding. Cells were counted after 3 days (lower panel).

FIG. 8 shows co-immunoprecipitation of plexin A-4 with FGFR1 or 2 or with VEGF receptor 2. The full length human plexin-A4 fused to a V5 tag was expressed in PAE (porcine aortic endothelial cells) with FGFR1 fused to a VSV tag or FGFR2 fused to a VSV tag. The cells were lysed and immuno-precipitation using V5 antibody was preformed. The western blot was subjected to VSV antibody in order to detect precipitation of FGFR1 or FGFR2 or an anti-VEGFR2 antibody in order to detect VEGF receptor 2.

FIGS. 9A-B are schematic illustrations showing the various Type-A Plexin interactions. FIG. 9A illustrates stimulatory signals through type-A plexin transduced via the interaction of plexin-A4 with semaphorin-6B, FGF receptor or VEGFR2. FIG. 9B illustrates inhibitory signals of type-A plexin transuded via the interaction of plexin-A4 with NP1 and sema3A or directly with sema6A. (The FGF receptor panel is adopted from Dickson et al. Breast Cancer Res 2000 2:191).

### DESCRIPTION OF SPECIFIC EMBODIMENTS OF THE INVENTION

The present invention, in some embodiments thereof, relates to high affinity molecules capable of binding a type A plexin receptor and uses of same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Neuropilin/plexin/semaphorin represent one of the complex signaling networks involved in axonal guidance and proliferative signaling.

Whilst reducing the present invention to practice, the present inventors uncovered that silencing of the type A plexin receptor family by specific shRNAs in endothelial cells leads to inhibition of bFGF induced proliferation but not to induction of apoptosis. Inhibition of the expression of each of these plexins resulted in reduced tube formation ability as compared to control cells. The present inventors have also shown that inhibition of the expression of the plexins results in reduced angiogenesis. An in-vitro angiogenesis assay was preformed using the shRNAs and revealed that those cells were almost unable to produce sprouts, an ability which is a critical step in angiogenesis.

In addition, inhibition of the expression of plexin-A1 or plexin-A4 in four different tumorigenic cell lines (A549, sw1614 and H460 lung cancer as well as in U87 glioma cells) also resulted in inhibition of cell proliferation. In order to determine if the decreased expression of plexin-A4 prevents tumor growth in-vivo, the present inventors implanted U87 cells subcutaneously in athymic nude mice. The cells that expressed lower amounts of plexin-A4, developed into significantly smaller tumors as compared to the control cells.

The present inventors were also able to demonstrate a similar effect when silencing the expression of semaphorin 6B, which might be responsible for a positive proliferative signal through plexin A receptors (e.g., A4), suggesting that inhibiting binding of these activators to plexin A4 would ultimately lead to the inhibition of basic FGF-dependent cell proliferation and angiogenesis. Accordingly, inhibition of the FGFR1 and semaphorin 6B axis, while retaining binding of neuropilin to plexin A (e.g., plexin A4) ultimately inhibits angiogenesis.

Thus, according to an aspect of the invention there is provided a high affinity molecule comprising a binding domain, wherein said binding domain binds a type A plexin receptor, blocks proliferative signals therefrom while maintaining inhibitory signals mediated by the type A plexin receptor.

As used herein “a high affinity molecule” refers to a naturally-occurring or synthetic molecule, which binds specifically a target protein molecule (e.g., plexin receptor) with an affinity higher than 10⁻⁶ M. Specific binding can be detected by various assays as long as the same assay conditions are used to quantify binding to the target versus control.

Examples of high affinity molecules which can be used in accordance with the present teachings, include, but are not limited to, an antibody, a peptide, an aptamer and a small molecule.

According to a specific embodiment the high affinity molecule is an antibody.

As used herein “a plexin A pathway activator” refers to a molecule that transduces a proliferative signal through the plexin A receptor. Examples include, but are not limited to, the type A plexin receptor, a co-receptor (e.g., FGFR, VEGFR-2), and a ligand (e.g., semaphorin 6B and 6D, bFGF and VEGF).

As used herein “a binding domain” refers to a chemical moiety having a general affinity towards the target molecule, e.g., the plexin pathway activator e.g., the type A plexin receptor. The general affinity is preferably higher than about, 10⁻⁶ M, 10⁻⁷ M, 10⁻⁸ M, 10⁻⁹ M, 10⁻¹⁰ M and as such is stable under physiological (e.g., in vivo) conditions.

Thus, the binding affinity of the binding domain to the plexin A pathway activator e.g., the type A plexin receptor is preferably higher than (i.e., at least) about, 10⁻⁴ M, 10⁻⁵ M, 10⁻⁶ M, 10⁻⁷ M, 10⁻⁸ M, 10⁻⁹ M, 10⁻¹⁰ M.

According to a specific embodiment the high affinity molecule is isolated, that is, isolated from a natural environment thereof i.e., where it is natively produced e.g., the human body.

As used herein a “Type A plexin receptor” refers to the semaphorin receptor family including, but not limited to, the following genes: PLXNA1,:PLXNA2,:PLXNA3,:PLXNA4A and:PLXNA4B.

According to a specific embodiment the plexin receptor, is plexin A4.

As mentioned hereinabove, the type A plexin family of receptors mediates proliferative signals either directly via semaphorin-6B or by still unidentified ligands that uses the plexin-A4\FGFR complex or Plexin-A4\VEGFR2 complex i.e., bFGF dependent or VEGFR-2. Accordingly, the high affinity molecule of the present invention blocks this proliferative signaling such as by interfering with binding to semaphorin (e.g., semaphorin 6B), FGFR receptors (e.g., GFR1, FGFR2), VEGFR2 or all.

Accordingly, as used herein “blocking” refers to at least 50%, 60%, 70%, 80%, 90%, 100% reduction in semaphorin and/or FGFR1 binding to the plexin receptor or co-receptor thereof. The reduction in binding may be a result from reduction in affinity or blocking of the binding site on the receptor. Binding can be assayed by Scatchard analysis for ligand-receptor binding and ligand competition binding assay which are well known in the art of biochemistry.

In addition, binding of the high affinity molecule does not interfere with inhibitory signals mediated by neuropilin binding to the receptor or binding of semaphorin 6A to the receptor. Thus according to a specific embodiment, neuropilin or semaphorin 6A binding to the receptor is maintained i.e., affinity to the receptor is essentially unchanged (or at least about 80% not changed).

A number of cell proliferation assays are known in the art such as for example thymidine incorporation assay and the MTT assay, each of which is well known in the art of cell biology.

As used herein “neuropilin” refers to the inhibitory co-receptors of type A plexin receptors. In fact, plexin serve as the signal transducing unit of the neuropilin.

According to a specific embodiment the neuropilin is neuropilin 1.

As used herein “semaphorin” refers to a semaphorin which mediates cell proliferation and angiogenesis by binding to a type A plexin receptor. According to a specific embodiment, the semaphorin is semaphorin 6B and the receptor is plexin A4 (co-receptor is FGFR1 and the ligand is bFGF).

According to a specific embodiment there is provided a high affinity molecule comprising a binding domain which binds a type A plexin receptor, wherein the binding domain inhibits proliferative signals through said type-A plexin receptor but does not interfere with binding of neuropilin to the receptor.

According to a more specific embodiment, the high affinity molecule comprising a binding domain which inhibits binding of semaphorin 6B and/or fibroblast growth factor receptor 1 (FGFR-1) to the type-A plexin receptor but does not interfere with binding of a neuropilin (e.g., neuropilin 1) to the type-A plexin receptor.

According to another more specific embodiment the semaphorin is semaphorin 6D which binds plexin Al (co-receptor is VEGFR2 and the ligand is VEGF).

According to a specific embodiment, the binding domain binds the type-A plexin receptor.

According to an embodiment of the invention the high affinity molecule binds an epitope on an extracellular domain of the Type A plexin receptor, the domain being selected from the group consisting of a sema domain (pfam number PF01403) and an IgG domain. It is well known that semaphorin binds the plexin receptor through the sema domain while FGFR1 interacts with the plexin through the IgG domain (this is a theoretical binding site—we have not confirmed yet). Sema domain corresponds to amino acids coordinates 24aa-507aa of SEQ ID NO: 1 and IgG like domains correspond to amino acid coordinates 858aa-1230aa of SEQ ID NO: 1 (plexin A4).

According to an embodiment of the invention, the high affinity molecule induces internalization/endocytosis of the plexin receptor upon binding thereto (see isolation of such antibodies in Examples 6 of the Examples section, which follows).

As mentioned, according to a specific embodiment, the molecule is an antibody.

The present invention further envisages an antibody comprising an antigen recognition domain which binds a type A plexin receptor, wherein the antibody induces internalization of said type A plexin receptor upon binding thereto.

It will be appreciated that internalization or endocytosis effectively down-regulates signaling via the receptor by removing it from the cell surface and rendering it inaccessible to extracellular ligands.

Methods of assaying ligand-induced receptor endocytosis are well known in the art and mostly employ cell surface labeling (e.g., radioactive or fluorescent) and monitoring the level of the signal over time in the presence and absence of the ligand (e.g., antibody). See for example L et al. Methods Mol. Biol. 2008 457:305-17; Sorkin 2008 Exp. Cell Res. 314:3093-106; and Barerford 2007 Adv. Drug Deliv. Rev. 59:748-58. Selection and characterization of antibodies that induce internalization of target receptors can be effected according to the method of Frans son and Borrebaeck as disclosed in Example 6 infra.

This antibody like those described above, can bind the sema and/or IgG domain of the plexin.

The term “antibody” as used in this invention includes intact molecules as well as functional fragments thereof, such as Fab, F(ab')2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab', the fragment of an antibody molecule that can be obtained by treating whole antibody with pepsin, followed by reduction, to yield an intact light chain and a portion of the heavy chain; two Fab' fragments are obtained per antibody molecule; (3) (Fab')2, the fragment of the antibody that can be obtained by treating whole antibody with the enzyme pepsin without subsequent reduction; F(ab')2 is a dimer of two Fab' fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab')2. This fragment can be further cleaved using a thiol reducing agent, and optionally a blocking group for the sulfhydryl groups resulting from cleavage of disulfide linkages, to produce 3.5S Fab' monovalent fragments. Alternatively, an enzymatic cleavage using pepsin produces two monovalent Fab' fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

As mentioned the antibody is designed to inhibit binding of semaphorin or a co-receptor thereof (e.g., FGFR1 or VEGFR-2) to plexin.

Accordingly, the antibody can be a bispecific antibody.

As used herein “bispecific” or “bifunctional” antibody, refers to an artificial hybrid antibody having two different heavy/light chain pairs and two different binding sites. Bispecific antibodies can be produced by a variety of methods including fusion of hybridomas. See e.g., Songsivilai and Lachmann (1990) Clin. Exp. Immunol. 79:315-321; Kostelny et al. (1992) J. Immunol. 148:1547-1553.

Thus, the bispecific antibody of some embodiments of the invention binds the type-A plexin receptor and at least one of the FGFR1 and the ligand (bFGF) as well as the semaphorin 6B.

Alternatively, the bispecific antibody binds distinct epitopes on the type-A plexin receptor.

For example, the antibody or the high affinity binds the sema domain 9pfam number PF01403) and the immunoglobulin domain.(pfam number 00047)

According to other embodiments, a bi-specific antibody of the invention binds to semphorin 6D and to the co-receptor VEGFR2 or the ligand VEGF. Humanized forms of any non-human (e.g., murine) antibodies are chimeric molecules of immunoglobulins, immunoglobulin chains or fragments thereof (such as Fv, Fab, Fab', F(ab').sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

As mentioned, the high affinity molecule may also be a peptide, such as a peptide derived from the extracellular portion of the plexin receptor and serving as a decoy by binding to semaphorin, the co-receptor or both.

Peptides (e.g., at least 10, 15, 20, 25 but no more than a 100 aa long) of the sema domain and/or the Ig (IPT) domain, as further described below may be used. Such peptides may be qualified for binding and sequestering semaphorin 6B or 6D or FGFR using biochemical assays known in the art such as ELISA, immunoprecipitation and the like.

The term “peptide” as used herein refers to a polymer of natural or synthetic amino acids, encompassing native peptides (either degradation products, synthetically synthesized polypeptides or recombinant polypeptides) and peptidomimetics (typically, synthetically synthesized peptides), as well as peptoids and semipeptoids which are polypeptide analogs, which may have, for example, modifications rendering the peptides even more stable while in a body or more capable of penetrating into cells.

Such modifications include, but are not limited to N terminus modification, C terminus modification, polypeptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided hereinunder.

Polypeptide bonds (—CO—NH—) within the polypeptide may be substituted, for example, by N-methylated bonds (—N(CH3)—CO—), ester bonds (—C(R)H—CO—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), polypeptide derivatives (—N(R)—CH2-CO—), wherein R is the “normal” side chain, naturally presented on the carbon atom.

These modifications can occur at any of the bonds along the polypeptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the polypeptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids (stereoisomers).

Tables 1 and 2 below list naturally occurring amino acids (Table 1) and non-conventional or modified amino acids (Table 2) which can be used with the present invention.

TABLE 1

| | Three-Letter | One-letter |
|---|---|---|
| Amino Acid | Abbreviation | Symbol |
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α ethylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmal | N-cyclooctylglycine | Ncoct |
| D-α-methylarginine | Dnmar | N-cyclopropylglycine | Ncpro |
| D-α-methylasparagine | Dnmas | N-cycloundecylglycine | Ncund |
| D-α-methylasparatate | Dnmas | N-(2,2-diphenylethyl)glycine | Nbhm |
| D-α-methylcysteine | Dnmcy | N-(3,3-diphenylpropyl)glycine | Nbhe |
| D-N-methylleucine | Dnmle | N-(3-indolylyethyl) glycine | Nhtrp |
| D-N-methyllysine | Dnmly | N-methyl-γ-aminobutyrate | Nmgabu |
| N-methylcyclohexylalanine | Nmchexa | D-N-methylmethionine | Dnmmet |
| D-N-methylornithine | Dnmor | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmva | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α thylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgl | N-(3-guanidinopropyl)glycine | Narg |
| D-N-methylglutamate | Dnmgl | N-(1-hydroxyethyl)glycine | Nthr |
| D-N-methylhistidine | Dnmhi | N-(hydroxyethyl)glycine | Nser |
| D-N-methylisoleucine | Dnmile | N-(imidazolylethyl)glycine | Nhis |
| D-N-methylleucine | Dnmle | N-(3-indolylyethyl)glycine | Nhtrp |
| D-N-methyllysine | Dnmly | N-methyl-γ-aminobutyrate | Nmgabu |
| N-methylcyclohexylalanine | Nmchexa | D-N-methylmethionine | Dnmmet |
| D-N-methylornithine | Dnmor | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmva | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α ethylvaline | Mtrp | L-α-methyltyrosine | Mtyr |
| L-α-methylleucine | Mval | L-N-methylhomophenylalanine | Nmhphe |
| | Nnbhm | | |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

The amino acids of the peptides of the present invention may be substituted either conservatively or non-conservatively.

The term “conservative substitution” as used herein, refers to the replacement of an amino acid present in the native sequence in the peptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)_{5—}COOH]—CO— for aspartic acid. Those non-conservative substitutions which fall under the scope of the present invention are those which still constitute a peptide having anti-bacterial properties.

As mentioned, the N and C termini of the peptides of the present invention may be protected by function groups. Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds.

These moieties can be cleaved in vivo, either by hydrolysis or enzymatically, inside the cell. Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a peptide of the present invention is protected, preferably with a methyl, ethyl, benzyl or substituted benzyl ester.

Examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O-CO—. Adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of

C-terminal protecting groups include —NH₂, —NHCH₃, —N(CH₃)₂, —NH(ethyl), —N(ethyl)₂ , —N(methyl) (ethyl), —NH(benzyl), —N(C1-C4 alkyl)(benzyl), —NH(phenyl), —N(C1-C4 alkyl) (phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

The peptides of the invention may be linear or cyclic (cyclization may improve stability). Cyclization may take place by any means known in the art. Where the compound is composed predominantly of amino acids, cyclization may be via N- to C-terminal, N-terminal to side chain and N-terminal to backbone, C-terminal to side chain, C-terminal to backbone, side chain to backbone and side chain to side chain, as well as backbone to backbone cyclization. Cyclization of the peptide may also take place through non-amino acid organic moieties comprised in the peptide.

The peptides of the present invention can be biochemically synthesized such as by using standard solid phase techniques. These methods include exclusive solid phase synthesis, partial solid phase synthesis methods, fragment condensation, classical solution synthesis. Solid phase polypeptide synthesis procedures are well known in the art and further described by John Morrow Stewart and Janis Dillaha Young, Solid Phase Polypeptide Syntheses (2nd Ed., Pierce Chemical Company, 1984).

Large scale peptide synthesis is described by Andersson Biopolymers 2000;55(3):227-50.

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

Recombinant techniques may also be used to generate the peptides of the present invention. To produce a peptide of the present invention using recombinant technology, a polynucleotide encoding the peptide of the present invention is ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present invention in the host cells.

The proteinecious high affinity molecules (e.g., peptides and antibodies) of the invention may be modified to increase bioavailability.

Thus, the peptides of the present invention may also comprise non-amino acid moieties, such as for example, hydrophobic moieties (various linear, branched, cyclic, polycyclic or hetrocyclic hydrocarbons and hydrocarbon derivatives) attached to the high affinity molecule; various protecting groups, especially where the compound is linear, which are attached to the compound's terminals to decrease degradation. Chemical (non-amino acid) groups present in the compound may be included in order to improve various physiological properties such; decreased degradation or clearance; decreased repulsion by various cellular pumps, improve immunogenic activities, improve various modes of administration (such as attachment of various sequences which allow penetration through various barriers, through the gut, etc.); increased specificity, increased affinity, decreased toxicity and the like.

Attaching the amino acid sequence component of the peptides/antibodies of the invention to other non-amino acid agents may be by covalent linking, by non-covalent complexion, for example, by complexion to a hydrophobic polymer, which can be degraded or cleaved producing a compound capable of sustained release; by entrapping the amino acid part of the peptide or antibody in liposomes or micelles to produce the final peptide of the invention. The association may be by the entrapment of the amino acid sequence within the other component (liposome, micelle) or the impregnation of the amino acid sequence within a polymer to produce the final high affinity molecule of the invention.

Other high molecular entities which can be used in accordance with the present teachings refer to aptamers and small molecules.

As used herein an “aptamer” refers to an oligonucleic acid (e.g., DNA or RNA) or peptide molecule that bind to a specific target molecule. In this case the aptamer is selected by binding to the plexin A receptor and/or activators thereof (similarly to antibody screening).

It will be appreciated that methods for identifying aptamers capable of specifically binding polypeptide targets are known in the art [e.g., U.S. Pat. No. 5,270,163, Ellington and Szostak (1990) Nature 346:818-822, Bock et al. (1992) Nature 255:564-566, Wang et al. (1993) Biochemistry 32:1899-1904, and Bielinska et al. (1990) Science 250:997-1000]. For example, U.S. Pat. No. 5,270,163 discloses a method referred to as SELEX (Systematic Evolution of Ligands by Exponential Enrichment) for the identification of nucleic acid ligands as follows. A candidate mixture of single-stranded nucleic acids having regions of randomized sequence is contacted with a target compound and those nucleic acids having an increased affinity to the target are partitioned from the remainder of the candidate mixture. The partitioned nucleic acids are amplified to yield a ligand enriched mixture. Bock and co-workers describe a method for identifying oligomer sequences that specifically bind target biomolecules involving complexation of the support-bound target molecule with a mixture of oligonucleotides containing random sequences and sequences that can serve as primers for PCR [Bock et al. (1992) Nature 255:564-566]. The target-oligonucleotide complexes are then separated from the support and the uncomplexed oligonucleotides, and the complexed oligonucleotides are recovered and subsequently amplified using PCR. The recovered oligonucleotides may be sequenced and subjected to successive rounds of selection using complexation, separation, amplification and recovery.

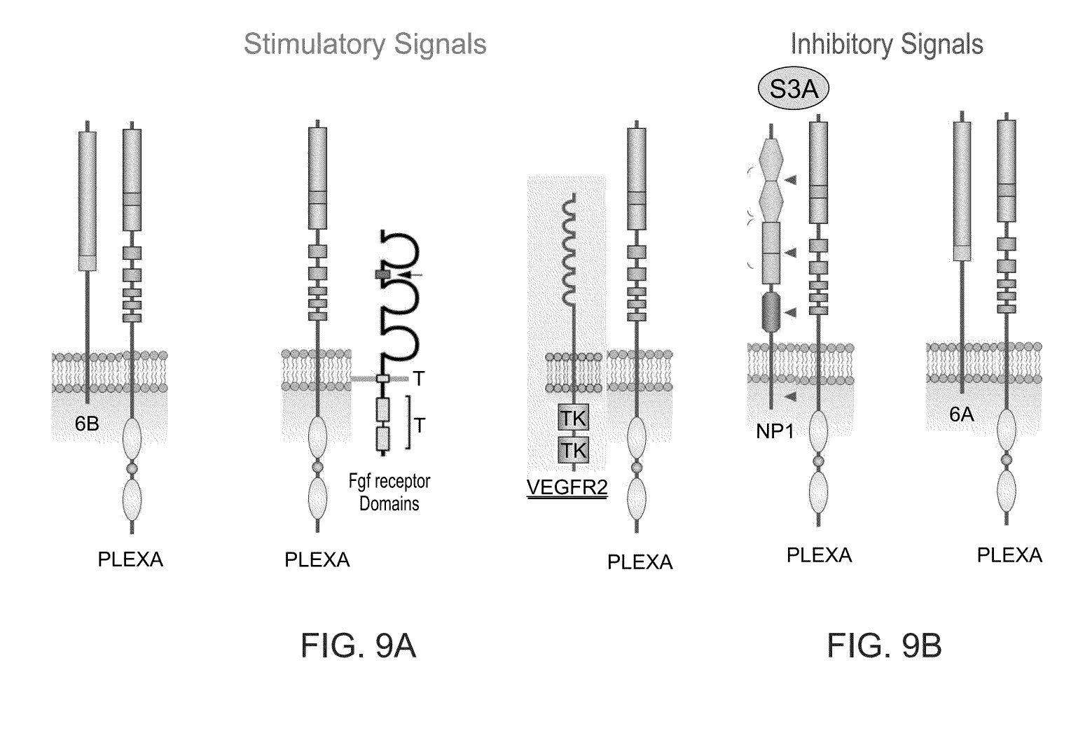

The realization that blockade of proliferative signaling via the type A plexin receptor while leaving the inhibitory cascade unaffected (i.e., via neuropilin 1) is therapeutically beneficial, allows the design of compositions in which at least two individual high affinity molecules each directed at a distinct activator of the proliferative plexin pathway (as described above e.g., VEGFR2, FGFR1, see FIGS. 9A-B) can be used.

Thus according to an aspect of the invention there is provided a composition-of-matter comprising at least two distinct high affinity molecules the at least two distinct high affinity molecules capable of binding and inhibiting proliferative signaling (as described hereinabove) from a plexin signaling molecule selected from the group consisting of a type A plexin receptor, a semaphorin, a co-receptor of said type A plexin receptor and a ligand of said co-receptor.

Thus, according to one embodiment, one high affinity molecule (e.g., an antibody) of the at least two distinct high affinity molecules binds sempaphorin (e.g., 6B or 6D) while the second high affinity molecule (e.g., an antibody) of the at least two distinct high affinity molecules binds bFGF.

According to another embodiment, one high affinity molecule (e.g., an antibody) of the at least two distinct high affinity molecules binds sempaphorin (e.g., 6B or 6D) while the second high affinity molecule (e.g., an antibody) of the at least two distinct high affinity molecules binds VEGF.

According to another embodiment, one high affinity molecule (e.g., an antibody) of the at least two distinct high affinity molecules binds a co-receptor of plexin (e.g., FGFR1) while the second high affinity molecule (e.g., an antibody) of the at least two distinct high affinity molecules binds VEGF.

According to another embodiment, one high affinity molecule (e.g., an antibody) of the at least two distinct high affinity molecules binds a type A plexin receptor while the second high affinity molecule (e.g., an antibody) of the at least two distinct high affinity molecules binds VEGF.

According to another embodiment, one high affinity molecule (e.g., an antibody) of the at least two distinct high affinity molecules binds bFGF, while the second high affinity molecule (e.g., an antibody) of the at least two distinct high affinity molecules binds VEGF.

According to another embodiment, one high affinity molecule (e.g., an antibody) of the at least two distinct high affinity molecules binds a first epitope on the type A plexin receptor (e.g., Ig domain), while the second high affinity molecule (e.g., an antibody) of the at least two distinct high affinity molecules binds a second epitope on the type A plexin receptor (e.g., sema domain).

Following is a non-limiting list of commercially available antibodies which can be used according to some embodiments of the invention. Further qualification of these antibodies e.g., for cell proliferation) can be effected according to the present teachings.

TABLE 3

| Target | Catalog Number | Company |
|---|---|---|
| VEGF (Avastin) | | Genentech |
| VEGF | Ab1319 | Abcam |
| | Ab68334 | Abcam |
| | Ab3109 | Abcam |
| | Ab119 | Abcam |
| | Ab27620 | Abcam |
| | sc-7269 | Santa cruz |
| | sc-73344 | Santa cruz |
| | sc-80442 | Santa cruz |
| VEGFR2 | Ab9530 | Abcam |
| | Ab42230 | Abcam |
| | Ab40669 | Abcam |
| | sc-74001 | Santa cruz |
| | sc-74002 | Santa cruz |
| | sc-57135 | Santa cruz |
| FGFR1 | Ab823 | Abcam |
| | Ab824 | Abcam |
| | Ab831 | Abcam |
| | Ab68419 | Abcam |
| | sc-57129 | Santa cruz |
| | sc-57130 | Santa cruz |
| | sc-73997 | Santa cruz |
| | sc-276 | Santa cruz |
| FGFR2 | Ab58201 | Abcam |
| | Ab89476 | Abcam |
| | sc-73738 | Santa cruz |
| | sc-6930 | Santa cruz |
| | sc-122 | Santa cruz |
| Sema6B | sc-67830 | Santa cruz |
| | sc-67831 | Santa cruz |
| | sc-74276 | Santa cruz |
| bFGF | Ab181 | Abcam |
| | Ab92337 | Abcam |
| | Ab18629 | Abcam |
| | Ab17505 | Abcam |
| | sc-74413 | Santa cruz |
| | sc-135905 | Santa cruz |
| | sc-71105 | Santa cruz |

As mentioned, the present inventors have realized that inhibition of signaling by plexin receptor effectively inhibits bFGF-dependent cell proliferation and angiogenesis.

Thus, according to another aspect of the invention there is provided a method of reducing angiogenesis in a tissue (e.g., as described hereinbelow), the method comprising contacting the tissue with the high affinity binding molecule (e.g., antibody) or a composition comprising same, as described hereinabove, thereby reducing angiogenesis in the tissue.

According to one embodiment, contacting with the tissue is effected ex-vivo.

According to one embodiment, contacting with the tissue is effected in-vivo.

As used herein “angiogenesis” refers to the growth of new blood vessels originating from existing blood vessels. Angiogenesis refers also to “vasculogenesis” which means the development of new blood vessels originating from stem cells, angioblasts or other precursor cells.

Angiogenesis can be assayed as described in the Examples section which follows or by measuring the total length of blood vessel segments per unit area, the functional vascular density (total length of perfused blood vessel per unit area), or the vessel volume density (total of blood vessel volume per unit volume of tissue).

The present invention further provides for a method of treating an angiogenesis-related disorder in a subject in need thereof, the method comprising administering to the subject a therapeutically effective amount of the high affinity biding molecule, thereby treating the angiogenesis-related disorder.

In a specific embodiment, the present invention specifically provides for a method of treating cancer in a subject in need thereof, the method comprising administering to the subject a therapeutically effective amount of the high affinity biding molecule, thereby treating cancer.

As used herein “subject” refers to a human or non-human (animal e.g., mammal) subject diagnosed with the disease.

As used herein “cancer” refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells.

By “disease” is meant any condition or disorder that damages or interferes with the normal function of a cell, tissue, or organ.

As used herein “angiogenesis related disorder” or “a disease associated with undesirable angiogenesis” (used interchangeably herein) refers to a clinical condition in which the processes regulating angiogenesis are disrupted and then pathology may result. Such a pathology affects a wide variety of tissues and organ systems. Diseases characterized by excess or undesirable angiogenesis are susceptible to treatment with the high affinity molecules described herein. The following provides a non-limiting list of such diseases.

Excess angiogenesis in numerous organs is associated with cancer and metastasis, including neoplasia and hematologic malignancies.

Angiogenesis-related diseases and disorders are commonly observed in the eye where they may result in blindness. Such disease include, but are not limited to, age-related macular degeneration, choroidal neovascularization, persistent hyperplastic vitreous syndrome, diabetic retinopathy, and retinopathy of prematurity (ROP).

A number of angiogenesis-related diseases are associated with the blood and lymph vessels including transplant arteriopathy and atherosclerosis, where plaques containing blood and lymph vessels form, vascular malformations, DiGeorge syndrome, hereditary hemorrhagic telangiectasia, cavernous hemangioma, cutaneous hemangioma, and lymphatic malformations.

Other angiogenesis diseases and disorders affect the bones, joints, and/or cartilage include, but are not limited to, arthritis, synovitis, osteomyelitis, osteophyte formation, and HIV-induced bone marrow angiogenesis.

The gastro-intestinal tract is also susceptible to angiogenesis diseases and disorders. These include, but are not limited to, inflammatory bowel disease, ascites, peritoneal adhesions, and liver cirrhosis.

Angiogenesis diseases and disorders affecting the kidney include, but are not limited to, diabetic nephropathy (early stage: enlarged glomerular vascular tufts).

Excess angiogenesis in the reproductive system is associated with endometriosis, uterine bleeding, ovarian cysts, ovarian hyperstimulation.

In the lung, excess angiogenesis is associated with primary pulmonary hypertension, asthma, nasal polyps, rhinitis, chronic airway inflammation, cystic fibrosis.

Diseases and disorders characterized by excessive or undesirable angiogenesis in the skin include psoriasis, warts, allergic dermatitis, scar keloids, pyogenic granulomas, blistering disease, Kaposi's sarcoma in AIDS patients, systemic sclerosis.

Obesity is also associated with excess angiogenesis (e.g., angiogenesis induced by fatty diet). Adipose tissue may be reduced by the administration of angiogenesis inhibitors

Excess angiogenesis is associated with a variety of auto-immune disorders, such as systemic sclerosis, multiple sclerosis, Sjogren's disease (in part by activation of mast cells and leukocytes). Undesirable angiogenesis is also associated with a number of infectious diseases, including those associated with pathogens that express (lymph)-angiogenic genes, that induce a (lymph)-angiogenic program or that transform endothelial cells. Such infectious disease include those bacterial infections that increase HIF-1 levels, HIV-Tat levels, antimicrobial peptides, levels, or those associated with tissue remodeling.

Infectious diseases, such as viral infections, can cause excessive angiogenesis which is susceptible to treatment with agents of the invention. Examples of viruses that have been found in humans include, but are not limited to, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronaviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Other angiogenesis-related disorders include, but are not limited to, hemangiomas, rheumatoid arthritis, atherosclerosis, idiopathic pulmonary fibrosis, vascular restenosis, arteriovenous malformations, meningiomas, neovascular glaucoma, psoriasis, angiofibroma, hemophilic joints, hypertrophic scars, Osler-Weber syndrome, pyogenic granuloma, retrolental fibroplasias, scleroderma, trachoma, vascular adhesion pathologies, synovitis, dermatitis, endometriosis, pterygium, wounds, sores, and ulcers (skin, gastric and duodenal).

The high affinity molecule(s) (e.g., antibody or antibodies) of the present invention can be administered to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the high effinity molecule (and optionally other active ingredients such as chemotherapy) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of an aggregate of cells having a similar structure and/or a common function. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue. The tissue may be a healthy tissue or a pathological tissue (e.g., a cancerous tissue or a tumor).

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (high affinity molecule) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue levels of the active ingredient which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

To increase therapeutic efficacy, the high affinity molecule may be administrated along with other drugs known for achieving a therapeutic effect. For example, chemotherapy may be administered for the treatment of cancer.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Silencing of Type A Plexins Inhibits Each of Endothelial cell Proliferation, Formation

### MATERIALS AND EXPERIMENTAL PROCEDURES

Quantification of plexin expression levels by Real-time PCR: Real-time PCR was preformed using Absolute Blue QPCR SYBR green mix according to the instructions of the manufacturer (Thermo Scientific). The following primers were used (Table 4, below):

TABLE 4

| Gene | Forward/ (SEQ ID NO:) | Reverse/ (SEQ ID NO:) |
|---|---|---|
| Plexin-A1 | TCCTGGTGGACCTCTCAAAC/ (SEQ ID | ACTGCACACAGCTCTCCACA/ (SEQ ID |
| | NO: 2) | NO: 3) |
| Plexin-A2 | CATCTCGTACTGGACCCCAC/ (SEQ ID | TTTACAACGGCTACAGCGTG/ (SEQ ID |
| | NO: 4) | NO: 5) |
| Plexin-A3 | ACCACGAAGGCACGGAAG/ (SEQ ID | AGCCAGCGGAGGGACAG/ (SEQ ID |
| | NO: 6) | NO: 7) |
| Plexin-A4 | TCTCAGTACAACGTGCTG/ (SEQ ID | TAGCACTGGATCTGATTGC/ (SEQ ID |
| | NO: 8) | NO: 9) |
| Sema3A | GGTTAACTAGGATTGTCTGTC/ (SEQ ID | GTGATCACATTGTTGGATTC/ (SEQ ID |
| | NO: 10) | NO: 11) |
| Sema6B | CTTACTTTGTCCATGCGGTG/ (SEQ ID | CACGTCGTTCTTGCACACTC/ (SEQ ID |
| | NO: 12) | NO: 13) |
| Actin | TTGCCGACAGGATGCAGAAGGA/ (SEQ ID | AGGTGGACAGCGAGGCCAGGAT/ (SEQ ID |
| | NO: 14) | NO: 15) |

Inhibition of plexin and semaphorin expression with shRNA expressing lentiviruses: Lentiviral ShRNA vectors carrying the shRNA sequences (Table 5, below) shown were purchased from Sigma Aldrich.

TABLE 5

| Gene | Sh-RNA sequence/SEQ ID NO: |
|---|---|
| Plexin-A1 | CCGGGCACTTCTTCACGTCCAAGATCTCGAGATCTTGGACG |
| | TGAAGAAGTGCTTTTTG/16 |
| Plexin-A2 | CCGGCGGCAATTTCATCATTGACAACTCGAGTTGTCAATGA |
| | TGAAATTGCCGTTTTTG/17 |
| Plexin-A3 | CCGGGCTGTATTTCTATGTCACCAACTCGAGTTGGTGACAT |
| | AGAAATACAGCTTTTTG/18 |
| Plexin-A4 | #1: CCGGGCAGATAAATGACCGCATTAACTCGAGTTAATG |
| | CGGTCATTTATCTGCTTTTTG/19 |
| | #2: CCGGCCTGACTTTGATATCTACTATCTCGAGATAGTA |
| | GATATCAAAGTCAGGTTTTTG/20 |
| Sema3A | CCGGCCCAATCTCAACACGATGGATCTCGAGATCCATCGTG |
| | TTGAGATTGGGTTTTTG/21 |
| Sema6B | CCGGTGGTTCAAAGAGCCTTACTTTCTCGAGAAAGTAAGGC |
| | TCTTTGAACCATTTTTG/22 |

### RESULTS

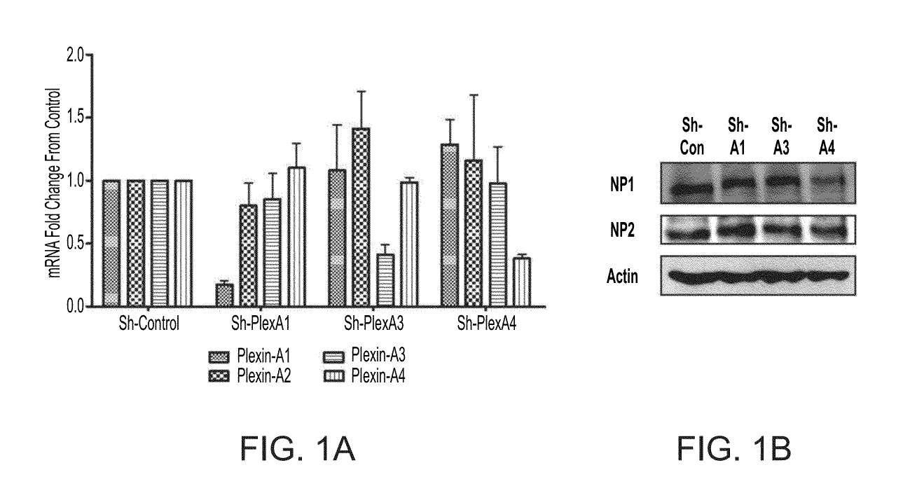

Silencing of Type A plexinS in HUVEC cells: Specific lentiviral shRNA encoding vectors were used to silence the expression of several endogenous type A plexin of HUVEC cells (FIGS. 1A-B). The specificity shRNA was examined by real-time PCR (FIG. 1A). The effect of the silencing on the expression the neuropilins which function as plexin co-receptors was also analyzed. Western blot analysis showed that silencing plexins does not affect neuropilin expression (FIG. 1B). It can be seen that the inhibitions of plexin expression were specific and none of the shRNAs inhibited neuropilin expression.

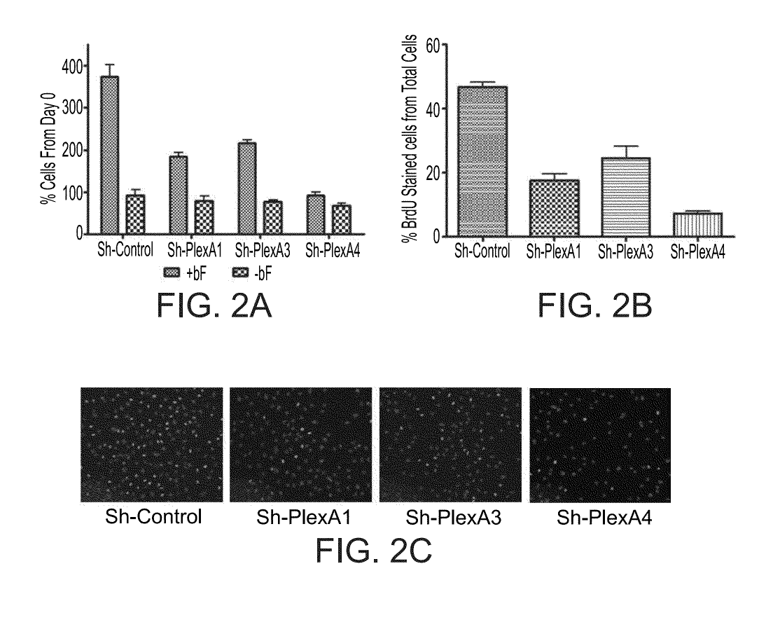

Anti-proliferative effects of silencing type A plexin in HUVEC cells: The proliferation rate of the silenced HUVEC cells was tested by seeding 2×10⁴ cells in 24 well plates coated with gelatin in the presence or absence of basic FGF (bF). The cells in each well were counted at the beginning of the experiment and at the end (3 days). It was observed that the silencing of all of the type A plexins resulted in a significant decrease in bFGF induced HUVEC proliferation (FIG. 2A). The results were confirmed by BrdU labeling experiments that label cells that enter the S phase of the cell cycle under the influence of bFGF (FIG. 2C). Again, inhibition of plexin expression resulted in less cells entering the cell cycle (FIG. 2B).

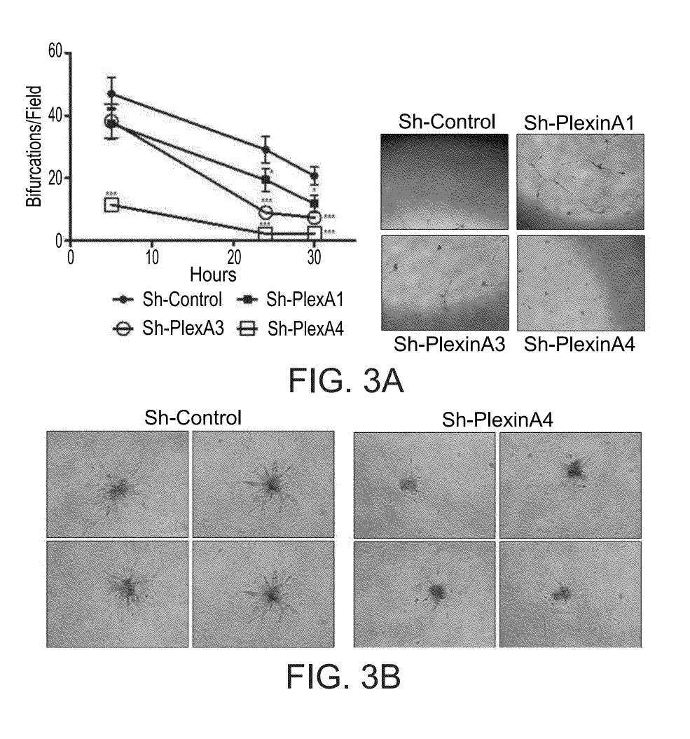

Silencing of type A plexins prevents the formation of blood vessel like tubes and HUVEC sprouting in-vitro: One of the indicators that endothelial cells (ECs) are functional and can form blood vessels in-vivo, is their ability to form tube like structures when seeded on matrigel in-vitro. Inhibition of the expression of the three type-A plexins studied resulted in inhibition of tube formation as observed after 30 hours (FIGS. 3A-B). The most potent effect was observed following inhibition of plexin-A4 expression but inhibition of the other plexins also had a significant inhibitory effect. A 3D sprouting assay which mimics the initial step of angiogenesis was done. In these assays spheroids of endothelial cells are embedded in collagen, stimulated with bFGF and allowed to sprout. Only cells in which the expression of plexin-A4 was inhibited were used in these assays because they showed the most potent inhibition of tube formation. Indeed, sprouting from spheroids containing HUVEC in which the expression of plexin-A4 was silenced was strongly inhibited (FIG. 3B).

### Example 2

### Silencing of Type A Plexin Decreases Tumor Cell Proliferation

### MATERIALS AND EXPERIMENTAL PROCEDURES

HUVEC and cancer cells proliferation assay: HUVEC cells were isolated and cultured as previously described [Kigel et al. 2008]. HUVEC infected with lentiviruses expressing a non-targeting shRNA (control cells) or HUVEC in which various plexins or semaphorins were silenced were seeded at a concentration of 2×10⁴ cells/well in 24 well dishes coated with PBS-gelatin, in the presence/absence of 5 ng/ml of bFGF. The number of adherent cells was then determined (time 0). Cells were counted after 3 days. The induction of proliferation was calculated as the fold increase in the number of cells relative to time 0. A similar protocol was used for cancer cells except that bFGF was omitted. Serum free proliferation assays using BHK-21 cells were performed using 0.1 ng/ml of bFGF as described [Kigel et al. 2008].

### RESULTS

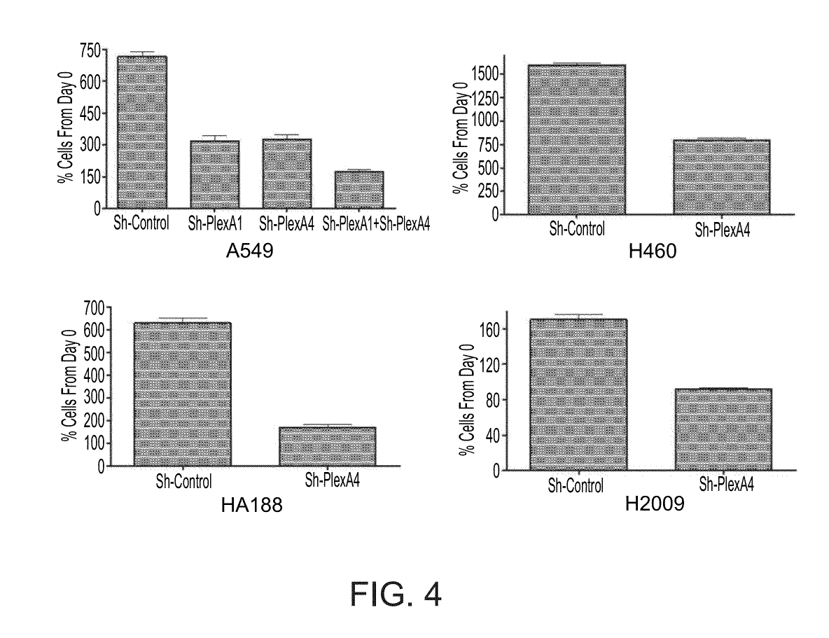

Silencing of plexin-A1 or plexin-A4 has an anti-proliferative effect on several lung cancer cell lines: The expression of plexins in several human tumor derived cell lines was inhibited. The expression of plexin-A1 and plexin-A4 was inhibited in A549 lung cancer cells. The inhibition resulted in significantly reduced proliferation of these cells. Inhibition of the expression of both plexins resulted in a stronger anti-proliferative effect then inhibition of the expression of each separately (FIG. 4).The effect was not specific to these cells alone since inhibition of plexin-A4 expression in several additional types of lung cancer derived cells also strongly inhibited their proliferation (FIG. 4).

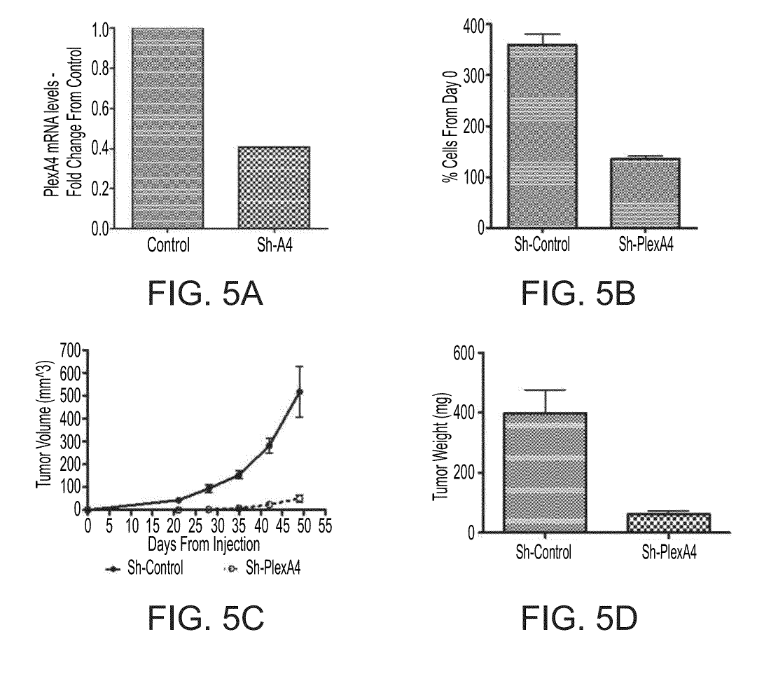

Silencing of plexin-A4 results in inhibition of tumor development: To find out if abolishing plexin-A4 in cancer cells other than lung cancer cell lines will have an anti-proliferative effect as well, plexin-A4 was silenced in U87 glioma cell lines (FIG. 5A). As in the case of the lung cancer cells, inhibition of plexin-A4 expression reduced the proliferation of the cells (FIG. 5B). Thereafter the effect of inhibition of plexin-A4 expression in U87 cells on the development of tumors from these cells was determined 1×10⁶ cells were implanted subcutaneously in athymic nude mice and tumor volume was measured once a week. At the end of the experiment the mice (n=10) were sacrificed and the tumors were weighted. It can be seen that inhibition of plexin-A4 expression results in a strong inhibition of tumor development (FIGS. 5C-D).

Thus, the expression of plexins in endothelial cells is likely to inhibit angiogenesis and tumor angiogenesis. Similarly, inhibition of the expression of plexins in tumor cells is likely to inhibit their proliferation and tumor development. Of these plexins, inhibition of plexin-A4 expression seems to produce the most potent effects.

### Example 3

### Inhibition of Sema6B Expression in HUVEC Mimics the Effects of Plexin-A4 Silencing

### MATERIALS AND METHODS—as described above

### RESULTS

Inhibition of sema6B expression in HUVEC mimics the effects of plexin-A4 silencing: The silencing of plexin-A4 expression in the endothelial cells and in the tumor cells results in inhibition of cell proliferation, suggesting that the inhibition disrupts an autocrine growth stimulatory signal conveyed by the plexin-A4 receptor. Class-3 semaphorins such as sema3A convey growth inhibitory signals. However, plexin-A4 also functions as a receptor for the class-6 semaphorins sema6A and sema6B {16173}. Interestingly, these two semaphorins are expressed in HUVEC as well as in lung and glioblastoma cells that respond to inhibition of plexin-A4 expression by inhibition of cell proliferation.

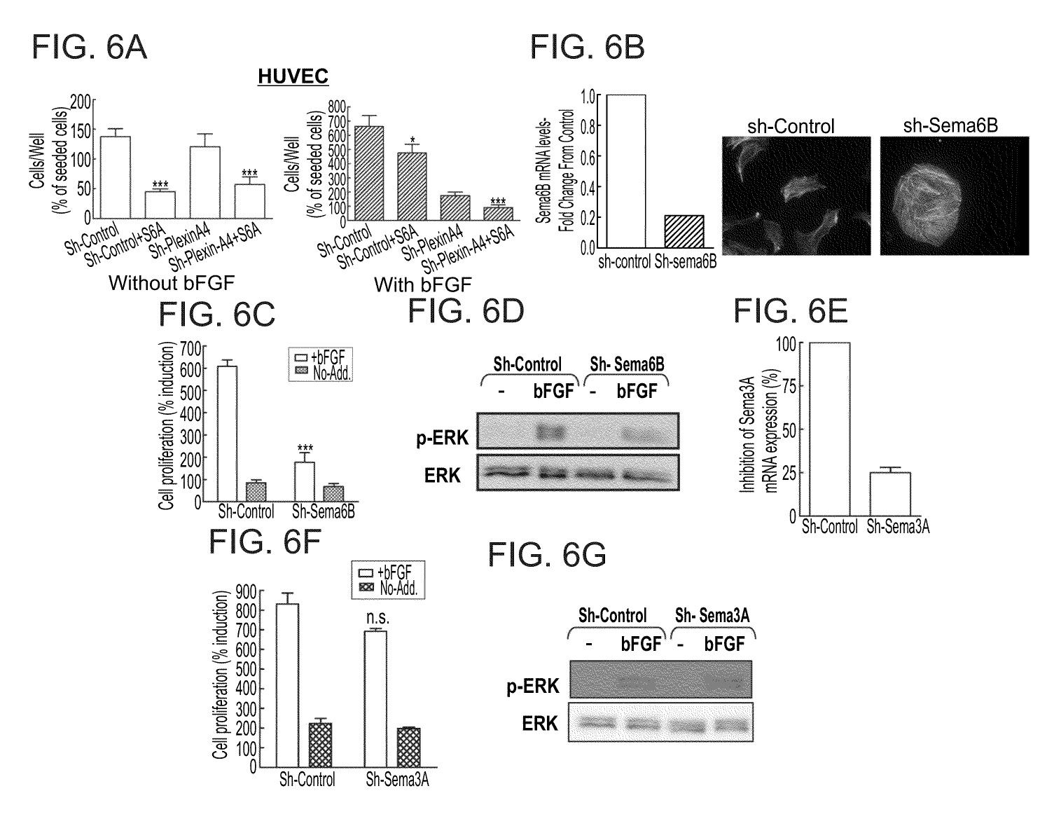

Stimulation of HUVEC with sema6A inhibited the bFGF induced proliferation of the HUVEC by ˜20% and in the absence of bFGF inhibited the survival of HUVEC by ˜70% (FIG. 6A). Furthermore, sema6A also inhibited the survival and the residual bFGF induced proliferative response in HUVEC in which plexin-A4 expression was silenced suggesting that in HUVEC sema6A transduces signals independently of plexin-A4 (FIG. 6A). Since sema6A functions in endothelial cells as an inhibitory factor it follows that it is unlikely that the silencing of plexin-A4 expression disrupts a sema6A induced autocrine growth stimulatory signaling loop. If that were the case, then such plexin-A4 silenced cells should have responded more vigorously than control cells to stimulation with bFGF.

Since HUVEC also produce sema6B, the role of sema6B in the formation of a plexin-A4 dependent growth stimulatory autocrine loop was determined. To that end, the expression of sema6B in the HUVEC was silenced. Interestingly, the silencing of the sema6B gene in the HUVEC resulted in a morphological change that was very reminiscent of the change produced in response to the silencing of plexin-A4 expression. Furthermore, silencing sema6B inhibited ˜85% of the mitogenic effect of bFGF (FIG. 6C) and to a similar extent inhibited bFGF induced phosphorylation of ERK1/2 (FIG. 6D). These results strongly suggest that the silencing of the plexin-A4 gene disrupts a sema6B dependent growth stimulating loop.

Since plexin-A4 also functions as a co-receptor for sema3A along with neuropilin-1, and because sema3A is also expressed by the endothelial cells, the effects of sema3A silencing on the behavior of the endothelial cells was tested. Sema3A functions as an inhibitor of angiogenesis, therefore it was expected that HUVEC silenced for sema3A expression would respond more vigorously to growth factors such as bFGF. However, cells in which sema3A expression was silenced (FIG. 1E) proliferated similarly to control cells in response to bFGF (FIG. 6F). Furthermore, there was also no difference between the level of ERK1/2 phosphorylation seen in response to stimulation with bFGF between the control cells and the sema3A silenced cells (FIG. 6G).

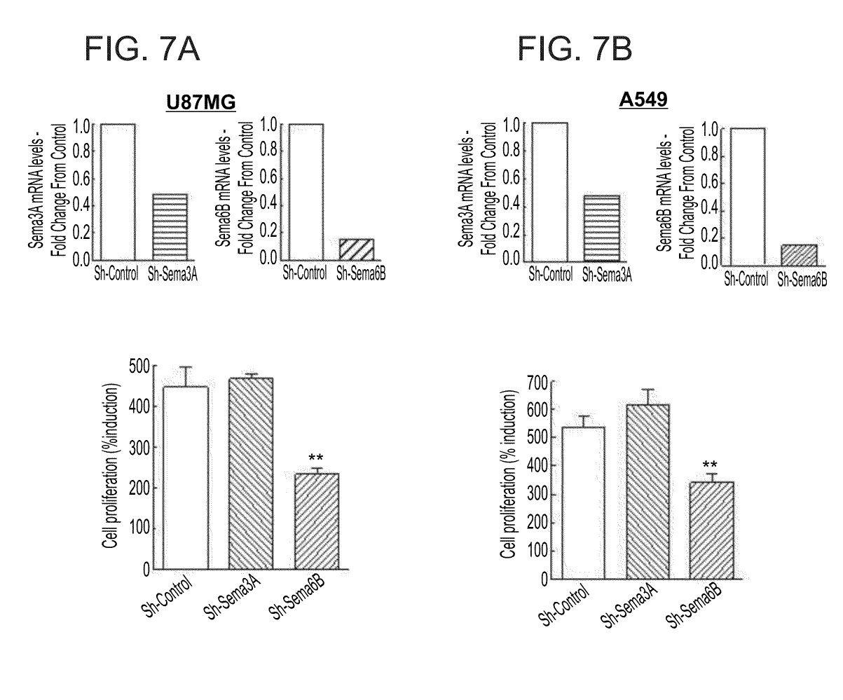

Inhibition of sema6B expression in tumor cells mimics the effects of plexin-A4 silencing: it inhibits their proliferation but does not affect their morphology: Inhibition of plexin-A4 expression in several types of tumor cells inhibits their proliferation. Since all of the tumor cells examined also express the sema6B and sema6A mRNAs encoding the known plexin-A4 ligands, the effects of silencing these genes on cell proliferation and cell shape were determined. Based on the present observations in the endothelial cells (see examples 1-2 above), it was hypothesized that the inhibitions observed when the expression of plexin-A4 is inhibited was a result of the disruption of an autocrine sema6B signaling loop. Both sema3A and sema6B expression were silenced in A549 and U87MG cells. Sema3A inhibition didn't have any effect on the proliferative rate of the cells. In contrast, silencing sema6B significantly inhibited the proliferation of these plexin-A4 dependent tumor cells (FIGS. 7A and B).

### Example 4

### Plexin A are Co-Receptors of FGFR1/2 Mediating FGF Proliferative Signals

### MATERIALS AND METHODS—as described herein.

### RESULTS

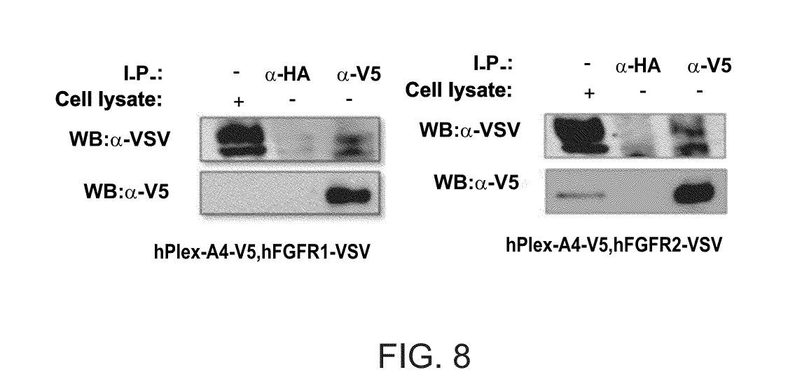

Plexin-A4 form a receptor complex with FGFR1 and FGFR2: The full length human plexin-A4 fused to a V5 tag was expressed in PAE (porcine aortic endothelial cells) with FGFR1 fused to a VSV tag or FGFR2 fused to a VSV tag. The cells were lysed and immuno-precipitation using V5 antibody was preformed. The western blot was subjected to VSV antibody in order to detect precipitation of FGFR1 or FGFR2 (FIG. 8).

Silencing plexin-A4 in endothelial cells inhibits their proliferation rate induced by bFGF. Alongside, it was shown for the first time that plexin-A4 can for a complex with the bFGF receptor, FGFR1. Thus, disruption of the plexin-A4\FGFR1 or any interaction between any type-A plexins and any FGF receptors interaction is likely to inhibit angiogenesis and tumor angiogenesis.

### Example 5

Plexin-A4 is known to tranduce sema3A and sema6A inhibitory signaling. Sema6A can bind directly to the plexin, while sema3A will form a complex with neuropilin-1, which acts as a co-receptor and then interact with plexin-A4.

Sema3A is known to inhibit angiogenesis and tumorgenesis, when ectopically expressed in various cancer cell lines (Kigel et al. 2008 PLoSONE. 3:e3287.). Sema3A binding results in inhibition of the density of blood vessels within the tumor, but it can also effect the anchorage independent growth of the cancer cells in-vitro. Kigel et al. supra, found that all of class-3 semaphorins have anti-angiogenic properties, but their ability to inhibit tumor progression is more dependent on the receptors (neuropilins) expressed on the tumor cell. Thus, breaking the neuropilin- 1\plexin-A4 complex will result in sema3A signal disruption and might give rise to an increased tumor progression (in the case the sema3A is present in the tumor micro-environment) or in a future therapy with class-3 semaphorin.

For these reasons an antibody that will block FGFR\Plexin-A4 or sema6B\Plexin-A4 complexes but will not block plexin-A4\neuropilin interaction is highly desired. Three IgG like domain that are present on the extra-cellular domain of FGFRs and on plexin-A4 may compose the hypothetical complexion site. The IgG like domain is also the dimerization site between FGFRs. Neuropiln-1 and sema6B form a complex with plexin-A4 on the same binding site that is called a “sema domain”. The site is about 500 amino acids. long and found on the N-terminal part of the extra-cellular region of the plexin (shown in FIG. 9). The sema domain can be found on all the semaphorin and plexins. Still, certain semaphorins bind to certain plexins, while others do not. For example, sema6B binds solely to plexin-A4, while sema6A can bind to both plexin-A4 and plexin-A2. Thus, although a high homology in the sema domain exists, there are still variations that distinguish between the complex formation capabilities of semaphorins and plexins.

The screening methodology for an antibody is performed using the phage display technique. Antibodies are screened against the entire extra-cellular portion of the human plexin-A4 (sema domain, PSI domains and IgG like domains (also termed IPT domains SEQ ID NO: 1)).

Antibodies that are found positive for binding the plexin-A4, are screened for their activity using in-vitro assays (proliferation and angiogenic assay). The antibodies that result in an inhibitory effect are characterized for their binding site to the plexin (epitope mapping) and for their ability to prevent the complex formation of plexin-A4 with the different receptors using assays developed in the lab. The extracellular part of plexin-A4 will be fused to an AP tag. The protein will be purified and will be used to various PAE (porcine aortic endothelial cells) that express the various tested receptors. One the plex-A4-AP will interact with the cells we would wash the cells and create a color reaction using the AP. We will use the appropriate antibodies to inhibit those complexes and thus there will be no color reaction of the AP.

### Example 6

### Isolation of Internalization-Inducing Antibodies

Isolation of phage-derived antibodies reactive to Plaexin-A4 which upon binding induce receptor-mediated internalization of the antibody/plexin A4 receptor is performed following the protocol of Fransson and Borrebaeck Methods in Molecular Biology, vol. 480: Macromolecular Drug Delivery Edited by: M. Belting Humana Press, a part of Springer Science+Business Media, LLC 2009. Plexin A4 (extra cellular portion) immunized mice are used for creating a phage library. This is done in order to find ScFV for specifically binding Plexin A4 and inducing internalization of same. The phage selection is done on whole cells stably expressing the antigen. To reduce the number of non-specific binders, the phage library is pre-incubated with the same cell line, not expressing the recombinant target antigen. In short, bound phages are allowed to internalize into the cells and are then rescued and enriched.

### EXPERIMENTAL PROCEDURES

Whole Cell Phage Selection

Negative Subtractor Cell Pre-selection

Subtractor non-target cells 10−500×106 cells) are precipitated by centrifugation at 4° C. (400×g, 5 min). The pellet-cells is dissolved in wash medium and the phage library (1×10¹³ cfu total phage) is added. The final volume is adjusted to 4 mL. The cell/phage mixture is incubated at 4° C. for 3 h on rotation. The cells are centrifuged and the supernatant containing the unbound phages is collected. The pellet is dissolved in 4 mL wash medium (RPMI 1640 cell culture medium, 10% (v/v) fetal calf serum, 50 mM HEPES buffer, pH 7.0, 2 mM EDTA). The cells are centrifuged again and and pooled with the supernatant.

The phages are precipitated by adding 25% PEG6000/2.5 M NaCl to the phage solution in a ratio of 1:4. The phages are incubated for 4 h or overnight at 4° C. The phages are pelleted by centrifugation at 4° C., 30 min, at 20,000×g.

The supernatant is discarded and the pellet is disolved in 1 mL of wash medium and stored at 4° C. until further use.

Positive Target Cell Selection

The target cells (10×10⁶ cells) are pelleted by centrifugation at 4° C. (400×g, 5 min).

The pellet is dissolved by adding the 1 mL solution containing the preselected library with another 1 mL wash medium to the tube that contained the pre-selected library to wash out the remaining phages. The cell/phage mixture is incubated at 4° C. for 1 h on rotation.

Antibodies Against Internalizing Antigens

To allow internalization of bound phages, the phage/cell suspension is transferred to a humidified atmosphere, containing 5% CO2, and incubated at 37° C. for 1 h. The cells are pelleted by centrifugation and the pellet is resuspended in 1 mL wash buffer.

The cell suspension is transferred to a 50-mL centrifuge tube containing 10 mL of 40% Ficoll, 2% BSA/PBS (without Ca2+) and centrifuged as described above.

The pellet is resuspended in mL PBS (with Ca²⁺) and PBS is added to a final volume of 10 mL. Cell pellet is generated as described above. Surface-bound phages are stripped by adding 5 mL stripping buffer and incubatde for 15 min. The cells are pelleted as described above. The cells are lysed by resuspending in 1 mL of 100 mM triethylamine and incubated for 5 min at room temperature The lysate is neutralized with 100 uL 1 M Tris-HCl, pH 8.3.

To rescue the selected phage, a TOP10F_culture (OD600=0.5) is infected by the output phages from the selection (30 min, 37° C.). The cells are spun down and resuspended in 1 mL of the supernatant. The cell suspension is plated on agar plates (amp/tet) and incubated at 37° C. overnight. The cells are harvested from the plates and suspended in 2×YT/amp/glu media and stored with 15% glycerol at −80° C.

New phage stocks are prepared from such pools of bacteria and the selection is repeated two to four times, depending on the output/input ratios.

After the last selection, individual colonies are picked, grown in culture, and stored as monoclonal glycerol stocks at −80° C.

### Example 7

### Antibody Binding Interference

In order to determine the ability of a candidate antibody to interfere with plexin-A4 ability to bind various co-receptors, a competitive ELISA is employed. 1n summary, ELISA plates are coated with the investigated co-receptor and the ability of plexin-A4 soluble receptor to bind the coated receptor in the presence of the antibody and analyzed.

The following reagents are used.

Recombinant human neuropilin-1 from R&D systems (cat: 3870-N1-025)

Recombinant human neuropilin-2 from R&D systems (cat: 2215-N2-025)

Recombinant mouse Plexin A1 from R&D systems (cat: 4309-PA-050)

Recombinant human VEGFR-2 from R&D systems (cat: 357-KD-050)

Recombinant human FGFR-1 from R&D systems (cat: 658-FR-050)

Recombinant human Semaphorin 6A from R&D systems (cat: 1146-S6-025)

Recombinant human Plexin A4 from R&D systems (cat: 5856-PA-050)

Recombinant human Semaphorin 6B from R&D systems (cat: 2094-S6-050)

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A high affinity molecule comprising a binding domain which binds a type-A plexin receptor, wherein said binding domain inhibits proliferative signals through said type-A plexin receptor but does not interfere with binding of a neuropilin or semaphorin 6A to said type-A plexin receptor.

2. A composition of matter comprising at least two distinct high affinity molecules said at least two distinct high affinity molecules capable of binding and inhibiting signaling from a plexin signaling molecule selected from the group consisting of a type A plexin receptor, a semaphorin, a co-receptor of said type A plexin receptor and a ligand of said co-receptor.

3. The high affinity molecule of claim 1, wherein said co-receptor is an FGFR or a VEGFR-.

4. The high affinity molecule of claim 1, wherein the high affinity molecule is selected from the group consisting of an antibody, a peptide, an aptamer and a small molecule.

5. The high affinity molecule of claim 1, wherein said type-A plexin receptor comprises Plexin-A4.

6. The high affinity molecule of claim 1, wherein said binding of said binding domain to said type-A plexin receptor comprises an affinity of at least 10⁻⁶ M.

7. The high affinity molecule of claim 4, wherein said antibody comprises a monoclonal antibody.

8. The high affinity molecule of claim 4, wherein said antibody comprises a bispecific antibody.

9. The high affinity molecule of claim 8, wherein said bispecific antibody binds said type-A plexin receptor and at least one of an FGFR and semaphorin 6B.

10. The high affinity molecule of claim 8, wherein said bispecific antibody binds a type-Al plexin receptor and at least one of VEGFR-2 and semaphorin 6D.

11. The high affinity molecule of claim 8, wherein said bispecific antibody binds to distinct epitopes on said type-A plexin receptor.

12. The high affinity molecule of claim 1, binding an epitope on an extracellular domain of said Type A plexin receptor, said extracellular domain being selected from the group consisting of a sema domain (pfam number PF01403) and an IgG domain.

13. The high affinity molecule of claim 1, wherein the high affinity molecule induces internalization of said plexin receptor.

14. An isolated antibody comprising an antigen recognition domain which binds a type A plexin receptor, wherein the antibody induces internalization of said type A plexin receptor upon binding thereto.

15. The high affinity molecule of claim 1, wherein said type-A plexin receptor is selected from the group consisting of Plxn-A1, Plxn-A2, Plxn-A3 and Plxn-A4.

16. The isolated antibody of claim 14, binding an epitope on an extracellular domain of said Type A plexin receptor, said domain being selected from the group consisting of a sema domain (pfam number PF01403) and an IgG domain.

17. A method of reducing angiogenesis in a tissue, the method comprising contacting the tissue with the high affinity molecule of claim 1, thereby reducing angiogenesis in the tissue.

18. The method of claim 17, wherein said contacting is effected ex-vivo.

19. The method of claim 17, wherein said tissue comprises a cancer tissue.

20. A method of treating an angiogenesis-related disorder in a subject in need thereof, the method comprising administering to the subject a therapeutically effective amount of the high affinity biding molecule of claim 1, thereby treating the angiogenesis-related disorder.

21. A method of treating cancer in a subject in need thereof, the method comprising administering to the subject a therapeutically effective amount of the high affinity binding molecule of claim 1, thereby treating cancer.

22. A pharmaceutical composition comprising a pharmaceutically acceptable carrier and as an active ingredient the high affinity molecule of claim 1.

23. The pharmaceutical composition of claim 22, further comprising a chemotherapeutic agent.


SEQUENCE LISTING (22 sequences)

SEQ ID NO: 1 - protein, 1230 aa, Homo sapiens
MKAMPWNWTCLLSHLLMVGMGSSTLLTRQPAPLSQKQRSFVTFRGEPAEGFNHLVVDERTGHIYLGAVNRIYKLSSDLKVLVTHETGPDEDNPKCYPPRIVQTCNEPLTTTNNVNKMLLIDYKENRLIACGSLYQGICKLLRLEDLFKLGEPYHKKEHYLSGVNESGSVFGVIVSYSNLDDKLFIATAVDGKPEYFPTISSRKLTKNSEADGMFAYVFHDEFVASMIKIPSDTFTIIPDFDIYYVYGFSSGNFVYFLTLQPEMVSPPGSTTKEQVYTSKLVRLCKEDTAFNSYVEVPIGCERSGVEYRLLQAAYLSKAGAVLGRTLGVHPDDDLLFTVFSKGQKRKMKSLDESALCIFILKQINDRIKERLQSCYRGEGTLDLAWLKVKDIPCSSALLTIDDNFCGLDMNAPLGVSDMVRGIPVFTEDRDRMTSVIAYVYKNHSLAFVGTKSGKLKKIRVDGPRGNALQYETVQVVDPGPVLRDMAFSKDHEQLYIMSERQLTRVPVESCGQYQSCGECLGSGDPHCGWCVLHNTCTRKERCERSKEPRRFASEMKQCVRLTVHPNNISVSQYNVLLVLETYNVPELSAGVNCTFEDLSEMDGLVVGNQIQCYSPAAKEVPRIITENGDHHVVQLQLKSKETGMTFASTSFVFYNCSVHNSCLSCVESPYRCHWCKYRHVCTHDPKTCSFQEGRVKLPEDCPQLLRVDKILVPVEVIKPITLKAKNLPQPQSGQRGYECILNIQGSEQRVPALRFNSSSVQCQNTSYSYEGMEINNLPVELTVVWNGHFNIDNPAQNKVHLYKCGAMRESCGLCLKADPDFACGWCQGPGQCTLRQHCPAQESQWLELSGAKSKCTNPRITEIIPVTGPREGGTKVTIRGENLGLEFRDIASHVKVAGVECSPLVDGYIPAEQIVCEMGEAKPSQHAGFVEICVAVCRPEFMARSSQLYYFMTLTLSDLKPSRGPMSGGTQVTITGTNLNAGSNVVVMFGKQPCLFHRRSPSYIVCNTTSSDEVLEMKVSVQVDRAKIHQDLVFQYVEDPTIVRIEPEWSIVSGNTPIAVWGTHLDLIQNPQIRAKHGGKEHINICEVLNATEMTCQAPALALGPDHQSDLTERPEEFGFILDNVQSLLILNKTNFTYYPNPVFEAFGPSGILELKPGTPIILKGKNLIPPVAGGNVKLNYTVLVGEKPCTVTVSDVQLLCESPNLIGRHKVMARVGGMEYSPGMVYIAP

SEQ ID NO: 2 - DNA, 20 bp, Artificial sequence
  Single strand DNA oligonucleotide
tcctggtggacctctcaaac

SEQ ID NO: 3 - DNA, 20 bp, Artificial sequence
  Single strand DNA oligonucleotide
actgcacacagctctccaca

SEQ ID NO: 4 - DNA, 20 bp, Artificial sequence
  Single strand DNA oligonucleotide
catctcgtactggaccccac

SEQ ID NO: 5 - DNA, 20 bp, Artificial sequence
  Single strand DNA oligonucleotide
tttacaacggctacagcgtg

SEQ ID NO: 6 - DNA, 18 bp, Artificial sequence
  Single strand DNA oligonucleotide
accacgaaggcacggaag

SEQ ID NO: 7 - DNA, 17 bp, Artificial sequence
  Single strand DNA oligonucleotide
agccagcggagggacag

SEQ ID NO: 8 - DNA, 18 bp, Artificial sequence
  Single strand DNA oligonucleotide
tctcagtacaacgtgctg

SEQ ID NO: 9 - DNA, 19 bp, Artificial sequence
  Single strand DNA oligonucleotide
tagcactggatctgattgc

SEQ ID NO: 10 - DNA, 21 bp, Artificial sequence
  Single strand DNA oligonucleotide
ggttaactaggattgtctgtc

SEQ ID NO: 11 - DNA, 20 bp, Artificial sequence
  Single strand DNA oligonucleotide
gtgatcacattgttggattc

SEQ ID NO: 12 - DNA, 20 bp, Artificial sequence
  Single strand DNA oligonucleotide
cttactttgtccatgcggtg

SEQ ID NO: 13 - DNA, 20 bp, Artificial sequence
  Single strand DNA oligonucleotide
cacgtcgttcttgcacactc

SEQ ID NO: 14 - DNA, 22 bp, Artificial sequence
  Single strand DNA oligonucleotide
ttgccgacaggatgcagaagga

SEQ ID NO: 15 - DNA, 22 bp, Artificial sequence
  Single strand DNA oligonucleotide
aggtggacagcgaggccaggat

SEQ ID NO: 16 - DNA, 58 bp, Artificial sequence
  Lentiviral expressed shRNA sequence
ccgggcacttcttcacgtccaagatctcgagatcttggacgtgaagaagtgctttttg

SEQ ID NO: 17 - DNA, 58 bp, Artificial sequence
  Lentiviral expressed shRNA sequence
ccggcggcaatttcatcattgacaactcgagttgtcaatgatgaaattgccgtttttg

SEQ ID NO: 18 - DNA, 58 bp, Artificial sequence
  Lentiviral expressed shRNA sequence
ccgggctgtatttctatgtcaccaactcgagttggtgacatagaaatacagctttttg

SEQ ID NO: 19 - DNA, 58 bp, Artificial sequence
  Lentiviral expressed shRNA sequence
ccgggcagataaatgaccgcattaactcgagttaatgcggtcatttatctgctttttg

SEQ ID NO: 20 - DNA, 58 bp, Artificial sequence
  Lentiviral expressed shRNA sequence
ccggcctgactttgatatctactatctcgagatagtagatatcaaagtcaggtttttg

SEQ ID NO: 21 - DNA, 58 bp, Artificial sequence
  Lentiviral expressed shRNA sequence
ccggcccaatctcaacacgatggatctcgagatccatcgtgttgagattgggtttttg

SEQ ID NO: 22 - DNA, 58 bp, Artificial sequence
  Lentiviral expressed shRNA sequence
ccggtggttcaaagagccttactttctcgagaaagtaaggctctttgaaccatttttg